(19) United States
(12) Patent Application Publication
(10) Pub. No.: US 2004/0267116 A1
(43) Pub. Date: Dec. 30, 2004

(54) System and method for providing an ultrasound recording environment

(76) Inventors: Ned Flood, Lowell, MA (US); Kevin Steen, Essex, MA (US)

Correspondence Address: Hayes Soloway P.C., 4th Floor, 175 Canal Street, Manchester, NH, 03101, US

(21) Appl. No.: 10/610,216

(22) Filed: Jun. 30, 2003

(51) Int. Cl. A61B 8/00

(52) U.S. Cl. 600/437; 600/443

(57) ABSTRACT

A system and method for providing an ultrasound recording environment is provided. Generally, the system contains an ultrasound machine and an ultrasound recording unit. The ultrasound recording unit contains a memory and a processor that is configured by the memory to perform the steps of: beginning recording of an entire ultrasound feed after an initiation to record; ending the recording, thereby providing a recorded ultrasound file; and filtering the recorded ultrasound file in accordance with a predefined region, thereby resulting in a filtered ultrasound file defined by the predefined region. Referring to the method, the method can be broadly summarized by the following steps: configuring the ultrasound recording unit; displaying a live ultrasound feed after being initiated to record by a user; beginning recording of the entire ultrasound feed after the initiation to record; ending the recording, thereby providing a recorded ultrasound file; and filtering the recorded ultrasound file in accordance with a predefined region defined during the configuration step, thereby resulting in a filtered ultrasound file defined by the predefined region.

### FIELD OF THE INVENTION

The present invention is generally related to medical devices, and more particularly is related to providing an ultrasound recording environment.

### BACKGROUND OF THE INVENTION

With medical advancements, it has become common for pregnant women to have different advanced medical examinations performed. One medical examination that is commonly performed on an expecting mother is a pre-natal diagnostic ultrasound. Ultrasounds allow a physician to determine medical status and condition of a child prior to birth. As is known by those having ordinary skill in the art, the ultrasound allows the physician to determine health and advancement of the child prior to birth (also referred to herein as a fetus) by measuring different parts of the fetus, and by accounting for all body parts that technically should be seen during performance of the ultrasound, specifically based upon advancement of the fetus. Ultrasounds also allow an expecting mother to view her child prior to birth. In fact, most women look forward to the ultrasound procedure since it is the first chance that the mother will have to view her child.

When properly performed, a prescribed pre-natal ultrasound is performed by a licensed ultrasound technician under prescription of a doctor and in a medical facility. Due to potential harm that may be caused by ultrasound waves, medical facilities maintain their ultrasound machines in accordance with a set maintenance schedule, thereby ensuring that emitted ultrasound waves are powerful enough to perform an ultrasound on a patient, yet not so powerful as to cause harm to the baby.

Recently, unlicensed individuals have offered an expecting mother the option of having a recording of an ultrasound performed. Unfortunately, these unlicensed individuals perform ultrasounds in a non-clinical setting and without the supervision or instruction of a doctor or licensed ultrasound technician. In fact, it is typical for the unlicensed individuals to only attend a short training course on how to use an ultrasound machine. An ultrasound machine located within the non-clinical setting is also typically not maintained in accordance with a regular maintenance schedule (e.g., weekly, or after each set number of ultrasounds is performed) to ensure that the ultrasound machine is working properly and not emitting dangerous levels of ultrasound waves. Therefore, these unlicensed individuals may pose a medical threat to the baby of the expecting mother.

Potential dangers associated with ultrasounds performed by unlicensed individuals are of such great concern that the unlicensed individuals are coming under increase pressure to cease operations from the Food and Drug Administration (FDA), the American Medical Association (AMA), the College of American Radiology, the Institute of Ultrasound in Medicine, and various state agencies. In fact, these dangers are of such concern that certain ultrasound machine manufactures have ceased the sale, lease, and/or rental of all ultrasound machines to any non-medically licensed individuals and/or organizations.

In addition to safety measures, recording of the ultrasound by unlicensed individuals is typically performed at a time different from the time the physician is performing the ultrasound for medical reasons. Specifically, the mother typically makes an appointment at a separate non-medical facility to have a recording of an ultrasound performed. Unfortunately, requiring the mother to travel to a separate facility that is equipped to record an ultrasound, in addition to the amount of time associated with waiting for an appointment to become available for recording of the ultrasound, is quite inconvenient.

There are also regulations, such as the health insurance portability and accountability act (HIPPA) privacy rule, which require permission from the individual having the ultrasound performed prior to recording of data associated with the ultrasound. Therefore, recording of the ultrasound is required to be performed without violating such rules.

Thus, a heretofore unaddressed need exists in the industry to address the aforementioned deficiencies, inconveniences, and inadequacies.

### SUMMARY OF THE INVENTION

Embodiments of the present invention provide a system and method for providing an ultrasound recording environment. Briefly described, in architecture, one embodiment of the system, among others, can be implemented as follows. The system contains an ultrasound machine and an ultrasound recording unit. The ultrasound recording unit contains a memory and a processor that is configured by the memory to perform the steps of: beginning recording of an entire ultrasound feed after an initiation to record; ending the recording, thereby providing a recorded ultrasound file; and filtering the recorded ultrasound file in accordance with a predefined region, thereby resulting in a filtered ultrasound file defined by the predefined region.

The present invention can also be viewed as providing methods for providing the ultrasound recording environment. In this regard, one embodiment of such a method, among others, can be broadly summarized by the following steps: configuring the ultrasound recording unit; displaying a live ultrasound feed after being initiated to record by a user; beginning recording of the entire ultrasound feed after the initiation to record; ending the recording, thereby providing a recorded ultrasound file; and filtering the recorded ultrasound file in accordance with a predefined region defined during the configuration step, thereby resulting in a filtered ultrasound file defined by the predefined region.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a block diagram of a recording system, including the present ultrasound recording unit, in accordance with a first exemplary embodiment of the invention.

FIG. 2 is a block diagram of the recording system where the ultrasound recording unit is located within the ultrasound machine.

FIG. 3 is a block diagram further illustrating the ultrasound recording unit located within the recording system of FIG. 1.

FIG. 4 is a block diagram further illustrating a computer located within the ultrasound recording unit of FIG. 3.

FIG. 5 is a flow chart illustrating configuration of the ultrasound recording unit prior to recording an ultrasound, in accordance with the first exemplary embodiment of the invention.

FIG. 6A and FIG. 6B are schematic diagrams providing examples of rendered images on the machine display of FIG. 1.

FIG. 7 is a flow chart illustrating the ultrasound recording procedure, in accordance with the first exemplary embodiment of the invention.

### DETAILED DESCRIPTION

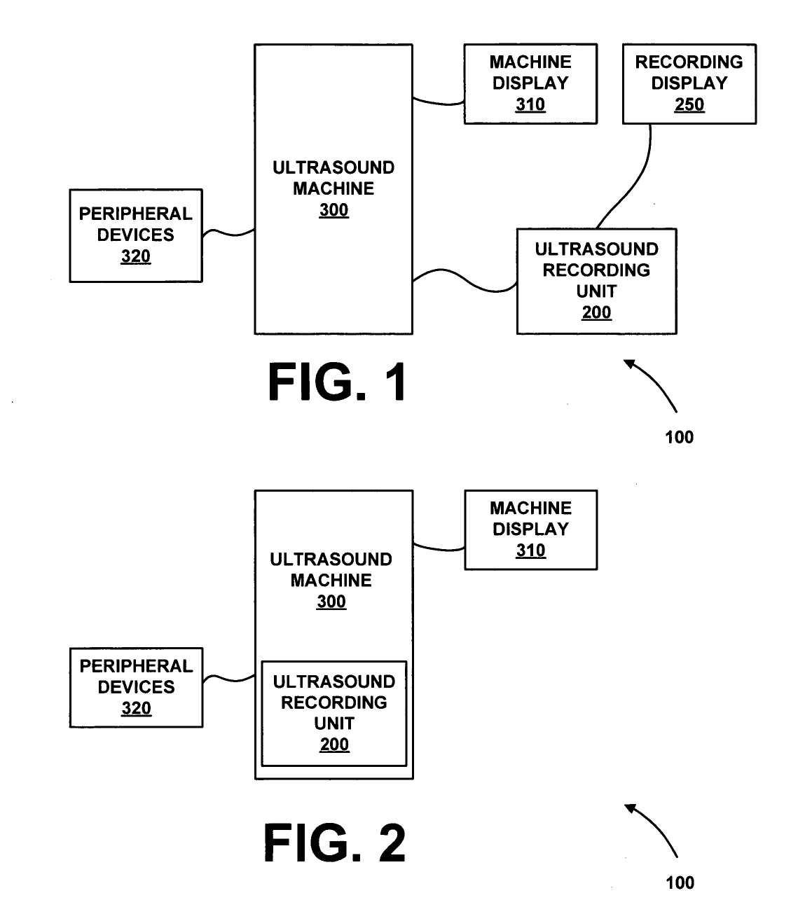

The present system and method provides an ultrasound recording environment by utilizing an ultrasound recording unit 200 (FIG. 1) that is capable of being directly connected to an ultrasound machine 300. It should be noted that, while the following refers to the use of an ultrasound for use on a pregnant woman, the present recording system may be used for other medical reasons.

FIG. 1 is a block diagram of a recording system 100, including the present ultrasound recording unit 200, in accordance with a first exemplary embodiment of the invention. As is shown by FIG. 1, the ultrasound recording unit 200 is connected to a recording display 250, such as, but not limited to, a computer monitor or a touch screen, the purpose of which is described in detail below. The ultrasound recording unit 200 is connected to the ultrasound machine 300 via, for example, a cable. Since one of ordinary skill in the art would know components of an ultrasound machine, a detailed description of the ultrasound machine 300 shown by FIG. 1, is not provided herein.

For purposes of properly describing the ultrasound recording unit 200, the following briefly describes certain known and associated parts of the ultrasound machine 300. A machine display 310, such as, but not limited to, a computer monitor or a touch screen, or even a television monitor, may be connected to the ultrasound recording unit 200. Alternatively, the machine display 310 may be located within the ultrasound machine 300.

In addition, the ultrasound machine 300 has a series of peripheral devices 320 connected thereto, such as, but not limited to, a keyboard and an ultrasound scanning device. As is known by one having ordinary skill in the art, the ultrasound scanning device is used to scan a pregnant woman during an ultrasound procedure. Typically, the ultrasound scanning device (not shown) has a button that, when initiated, causes the ultrasound scanning device to begin transmitting ultrasound waves. Alternatively, some ultrasound scanning devices do not have a button on the device to initiate scanning. Instead, these ultrasound scanning devices may be initiated via the keyboard (i.e., peripheral device 320) attached to the ultrasound machine 300, or a different initiation means. In accordance with a second exemplary embodiment of the invention, the ultrasound recording unit 200 may use the machine display 310, instead of requiring the separate recording display 250. Therefore, anything described below as being displayed on the recording display 250, may instead be displayed on the machine display 310. In addition, all displaying may be provided exclusively on the recording display 250.

It should be noted that, in accordance with an alternative embodiment of the invention, the ultrasound recording unit 200 may be located within the ultrasound machine 300. FIG. 2 is a block diagram of the recording system 100 where the ultrasound recording unit 200 is located within the ultrasound machine 300. Note that when the ultrasound recording unit 200 is located within the ultrasound machine 300, it is preferable that the machine display 310 be used by both the ultrasound recording unit 200 and the ultrasound machine 300. For exemplary purposes, the following description assumes that the ultrasound recording unit 200 is located separate from the ultrasound machine 300, as is shown by FIG. 1.

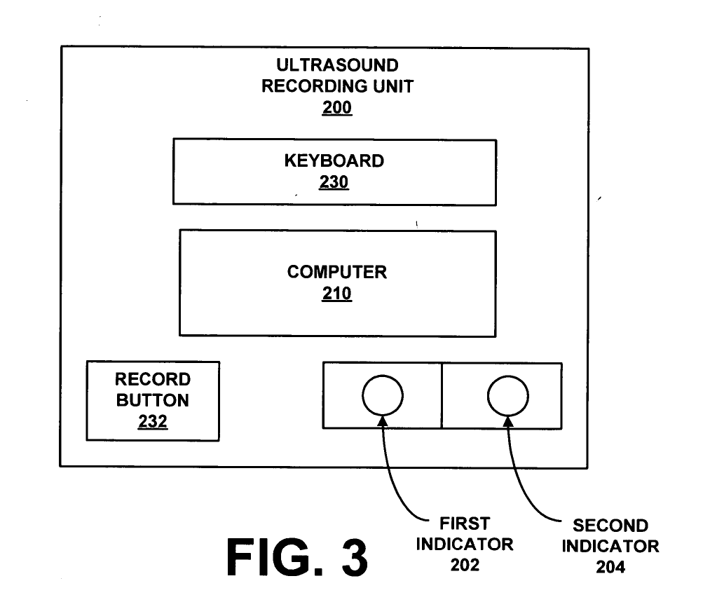

FIG. 3 is a block diagram further illustrating the ultrasound recording unit 200 located within the recording system 100 of FIG. 1, in accordance with the first exemplary embodiment of the invention. As is shown by FIG. 3, the ultrasound recording unit 200 has a series of indicators 202, 204, specifically, a first indicator 202, and a second indicator 204. The first indicator 202 and the second indicator 204 are preferably light emitting diodes (LEDs), wherein the first indicator 202 is a red LED and the second indicator 204 is a green LED. A detailed description of use of the indicators 202, 204 is provided below. It should be noted that the first indicator 202 and the second indicator 204 need not be LEDs, but instead, may be any devices that are capable of indicating to a user of the present recording system 100 that certain functions have begun, and when the functions have ended. In fact, a single LED may instead be used, where the beginning of certain defined functions, as described below, is indicated by lighting of the LED, and ending of the functions is indicated by the lit LED turning off. Alternatively, the machine display 310 or the recording display 250 may be used to indicate to a user of the present recording system 100 that certain functions have begun, and when the functions have ended.

As is shown by FIG. 3, the ultrasound recording unit 200 also contains a computer 210, a keyboard 230, and a record button 232. It should be noted that the keyboard 230 is used to allow a user of the ultrasound recording unit 200 to communicate commands and enter information to the ultrasound recording unit 200. Alternatively, the keyboard 230 may be located separate from the ultrasound recording unit 200 or a different means of communicating commands to the ultrasound recording unit 200 may be used. As an example, the ultrasound recording unit 200 may instead contain functionality to allow inputting of commands and information to the ultrasound recording unit 200 via speech recognition, or a remote controller.

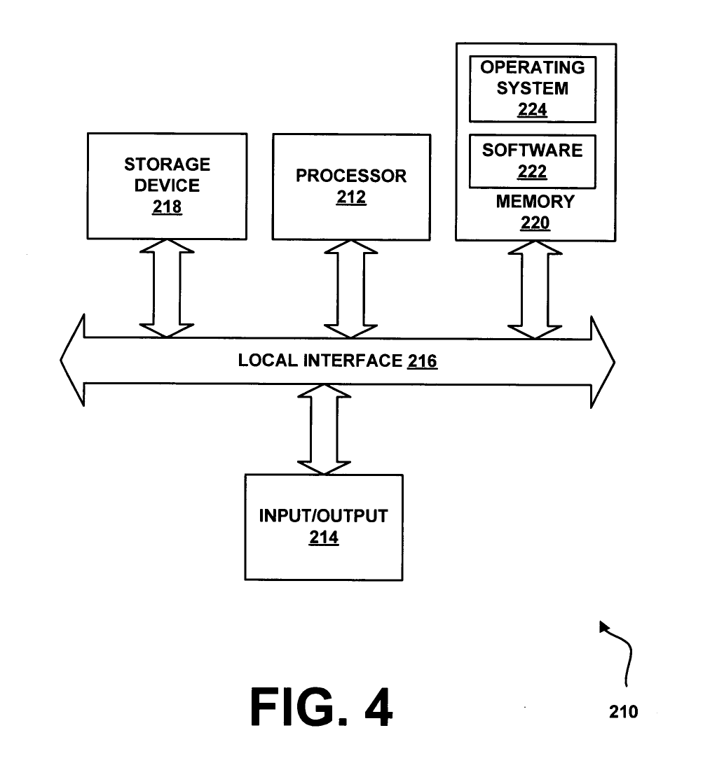

Functionality for allowing the ultrasound recording unit 200 to perform different actions, so as to provide the present recording system 100, may be provided by software 222 (FIG. 4) stored within the computer 210 portion of the ultrasound recording unit 200. Further description of these actions is provided hereafter. Therefore, a portion of the present recording system 100 may be implemented in software (e.g., firmware), hardware, or a combination thereof. As is mentioned above, in the currently contemplated best mode, at least a portion of the ultrasound recording unit 200 is implemented in software, as an executable program, and is executed by a special or general purpose digital computer, a personal computer (PC; IBM-compatible, Apple-compatible, or otherwise), such as the computer 210 of FIG. 3, workstation, minicomputer, or mainframe computer.

An example of the computer 210 located within the ultrasound recording unit 200 that can implement functionality to provide the present recording system 100 is shown by FIG. 4. Specifically, FIG. 4 is a block diagram further illustrating the computer 210 located within the ultrasound recording unit 200 of FIG. 3.

Generally, in terms of hardware architecture, as shown in FIG. 4, the computer 210 contains a processor 212, memory 220, and one or more input and/or output (I/O) ports 214 that are communicatively coupled via a local interface 216. The local interface 216 can be, for example, but not limited to, one or more buses or other wired or wireless connections, as is known in the art. The local interface 216 may have additional elements, which are omitted for simplicity, such as controllers, buffers (caches), drivers, repeaters, and receivers, to enable communications. Further, the local interface 216 may include address, control, and/or data connections to enable appropriate communications among the aforementioned components.

The I/O ports 214 may allow peripheral devices to connect to the ultrasound recording unit 200. The peripheral devices may include input devices, for example but not limited to, a keyboard, a mouse, etc. Furthermore, the peripheral devices may include output devices, for example but not limited to, a printer, display, etc. Specifically, such peripheral devices may include the record button 232, the first indicator 202 and the second indicator 204, and the keyboard 230. The I/O ports 214 may also provide an interface for allowing the ultrasound recording unit 200 to be connected the ultrasound machine 300. In addition, the I/O ports 214 may allow the recording display 250 to be connected to the ultrasound recording unit 200. Use of the recording display 250 by the ultrasound recording unit 200 is described in detail below. If the I/O ports 214 include an interface, the interface may be an S-video input and output, a serial or parallel connector, a micro D connector, a cable ready connector, or any other interface. It should be noted that other peripheral devices that may connect to the ultrasound recording unit 200 via the I/O ports 214 may include devices that communicate both inputs and outputs, for instance but not limited to, a modulator/demodulator (modem; for accessing another device, system, or network), a radio frequency (RF) or other transceiver, a telephonic interface, a bridge, a router, etc. Of course, other peripheral devices my also be connected to the I/O ports 214.

The processor 212 is a hardware device for executing system software 222, particularly that stored in the memory 220. The processor 212 can be any custom made or commercially available processor, a central processing unit (CPU), an auxiliary processor among several processors associated with the computer 210, a semiconductor based microprocessor (in the form of a microchip or chip set), a macroprocessor, or generally any device for executing software instructions. Examples of suitable commercially available microprocessors are as follows: a PA-RISC series microprocessor from Hewlett-Packard Company, an 80×86 or Pentium series microprocessor from Intel Corporation, a PowerPC microprocessor from IBM, a Sparc microprocessor from Sun Microsystems, Inc, or a 68xxx series microprocessor from Motorola Corporation.

The memory 220 can include any one or combination of volatile memory elements (e.g., random access memory (RAM, such as DRAM, SRAM, SDRAM, etc.)) and nonvolatile memory elements (e.g., ROM, hard drive, tape, CDROM, etc.). Moreover, the memory 220 may incorporate electronic, magnetic, optical, and/or other types of storage media. It should be noted that the memory 220 may have a distributed architecture, where various components are situated remote from one another, but can be accessed by the processor 212.

Software located in the memory 220 may include one or more separate programs, each of which comprises an ordered listing of executable instructions for implementing logical functions. In the example of FIG. 4, the software located in the memory 220 is system software 222 that at least includes logic defining functionality that provides the present recording system 100. The memory 220 may also contain a suitable operating system (O/S) 224. A nonexhaustive list of examples of suitable commercially available operating systems 224 is as follows: (a) a Windows operating system available from Microsoft Corporation; (b) a Netware operating system available from Novell, Inc.; (c) a Macintosh operating system available from Apple Computer, Inc.; (e) a UNIX operating system, which is available for purchase from many vendors, such as the Hewlett-Packard Company, Sun Microsystems, Inc., and AT&T Corporation; (d) a LINUX operating system, which is freeware that is readily available on the Internet; (e) a run time Vxworks operating system from WindRiver Systems, Inc.; or (f) an appliance-based operating system, such as that implemented in handheld computers or personal data assistants (PDAs) (e.g., PalmOS available from Palm Computing, Inc., and Windows CE available from Microsoft Corporation). The operating system 224 may essentially control the execution of other computer programs, such as the system software 222, and provides scheduling, input-output control, file and data management, memory management, and communication control and related services.

The system software 222 is a source program, executable program (object code), script, or any other entity comprising a set of instructions to be performed. When a source program, then the program needs to be translated via a compiler, assembler, interpreter, or the like, which may or may not be included within the memory 220, so as to operate properly in connection with the O/S 224. Furthermore, the system software 222 can be written as (a) an object oriented programming language, which has classes of data and methods, or (b) a procedure programming language, which has routines, subroutines, and/or functions, for example but not limited to, C, C++, Pascal, Basic, Fortran, Cobol, Perl, and Java.

If the computer 210 is a personal computer (PC), workstation, or the like, the memory 220 may further contain software that includes a basic input output system (BIOS) (omitted for simplicity). The BIOS is a set of essential software routines that initialize and test hardware at startup, start the operating system 224, and support the transfer of data among the hardware devices. The BIOS is stored in ROM so that the BIOS can be executed when the computer 210 is activated.

When the computer 210 is in operation, the processor 212 is configured to execute the system software 222 stored within the memory 220, to communicate data to and from the memory 220, and to generally control operations of the computer 210 pursuant to the system software 222.

The computer 210 also contains a storage device 218 for storing video during recording (as is explained in detail below). The storage device 218 may be a computer readable medium that is removable, stationary, or stationary with a removable computer readable medium located therein that is removable. The storage device 218 may be an electronic, magnetic, optical, or other physical device or means that can contain or store video and/or a computer program, for use by or in connection with a computer 210. In the context of this document, a “computer-readable medium” can be any means that can store, communicate, propagate, or transport video or software for use by or in connection with an instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium may include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical).

In an alternative embodiment, where the recording system 100 is implemented entirely in hardware, the recording system 100 can be implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

Configuration

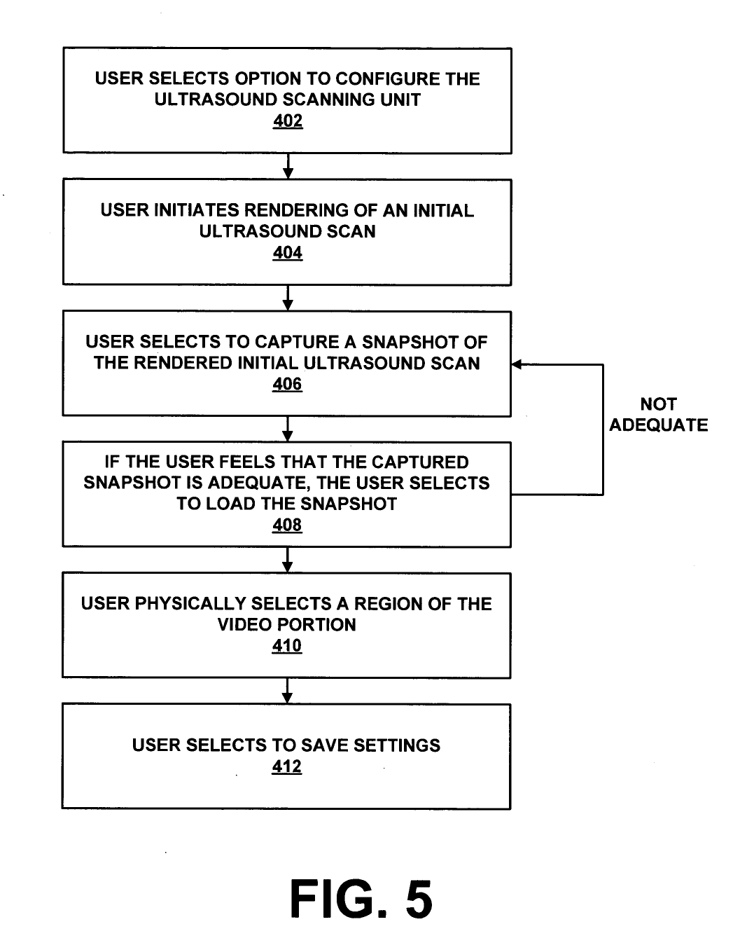

Use of the ultrasound recording unit 200 may require configuration prior to use, as is described in detail below. FIG. 5 is a flow chart illustrating configuration of the ultrasound recording unit 200 prior to recording an ultrasound, in accordance with the first exemplary embodiment of the invention. It should be noted that any process descriptions or blocks in flow charts should be understood as representing modules, segments, portions of code, or steps that include one or more instructions for implementing specific logical functions in the process, and alternative implementations are included within the scope of the present invention in which functions may be executed out of order from that shown or discussed, including substantially concurrently or in reverse order, depending on the functionality involved, as would be understood by those reasonably skilled in the art of the present invention.

As is shown by block 402, the ultrasound recording unit 200 displays an option to configure the unit 200 on the recording display 250. Preferably, the option to configure the ultrasound recording unit 200 is provided as an icon on the recording display 250, that may be selected by the user of the unit 200. Selection of the configuration icon may be performed via use of different peripheral devices, such as a mouse, keyboard, or other devices. For exemplary purposes, the following assumes that the user uses the keyboard 230 to select the configuration icon.

As is shown by block 404, the user of the ultrasound recording unit 200 may then initiate rendering of an initial ultrasound scan by using the ultrasound scanning device, thereby providing a real-time rendered ultrasound on the machine display 310 (FIG. 1). Alternatively, the user of the ultrasound recording unit 200 may initiate rendering of an initial scan by simply placing a lubricant, or gel, on the ultrasound scanning device. As is known by those having ordinary skill in the art, placing gel on the ultrasound scanning device results in the real-time rendered ultrasound not being clear since no tissue or dense matter is being scanned. It should be noted that it may not be necessary for the user to render an initial ultrasound scan, as is further explained below.

Typically, the machine display 310 (FIG. 1) displays results of the initial ultrasound scan, or, in the case of using gel and not placing the ultrasound scanning device on the pregnant woman, a rendered image that is not clear, within a video portion (explained below) of the machine display 310 (FIG. 1). The machine display 310 (FIG. 1) also displays medical data in different predefined regions of the machine display 310 (FIG. 1), referred to herein as the data portion (explained below) of the machine display 310 (FIG. 1). Unfortunately, most individuals that are not medical personnel, cannot read and/or understand the medical data, and therefore, have no need for the medical data. In addition, it should be noted that, as has been previously mention, there are regulations on the recording of medical data, therefore, the present ultrasound recording unit 200 filters out the medical data, as is explained in detail below.

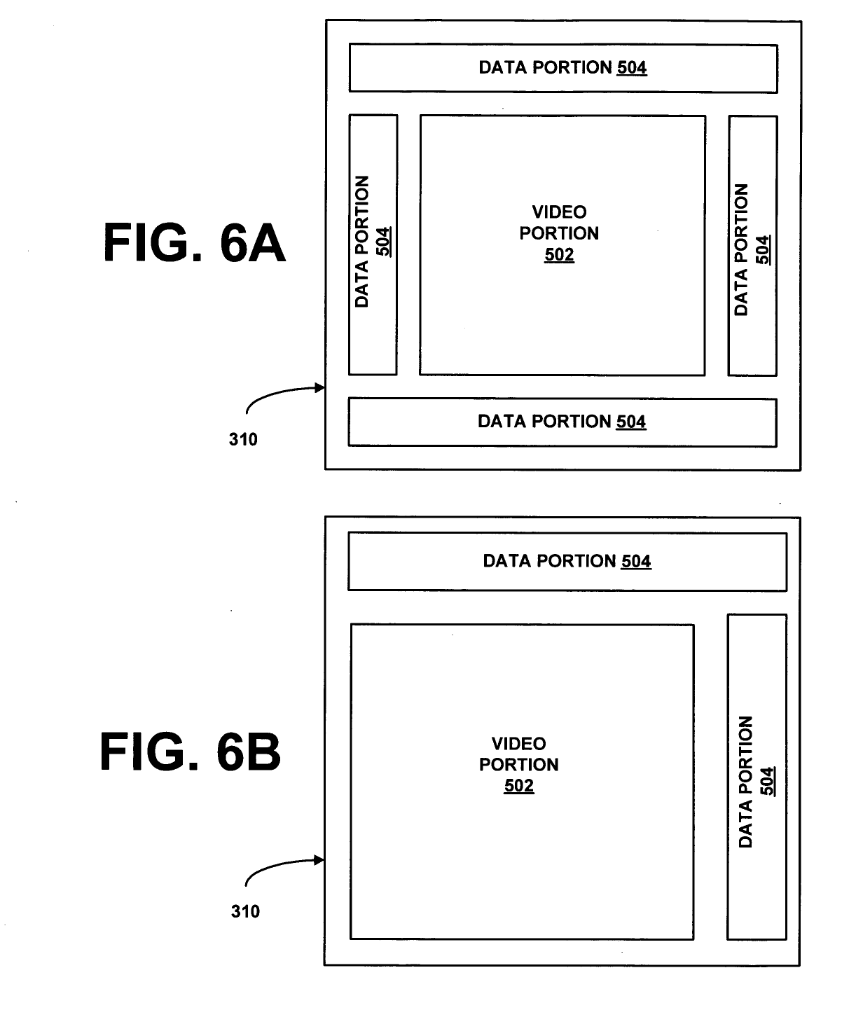

For exemplary purposes, FIG. 6A and FIG. 6B are schematic diagrams providing examples of rendered images on the machine display 310, including the video portion 502 and the data portion 504. As is shown by FIG. 6A and FIG. 6B, the machine display 310 may have multiple data portions 504 that may be located at different portions of the machine display 310. In addition, it is typical for the machine display 310 to have one video portion 502 that displays an ultrasound while a physician is scanning a pregnant woman.

Returning to FIG. 5, the user may then select an option, provided by the ultrasound scanning unit 200 and displayed via the recording display 250, to capture a single frame, or snapshot, of the initial ultrasound scan (block 406). It should be noted that the captured snapshot is preferably displayed on the recording display 250 and includes data portions 504 and video portions 502 that were initially displayed on the machine display 310 (FIG. 1). If the user feels that the captured snapshot is adequate, the user may select to load the captured snapshot, into the memory 220 (block 408) so as to temporarily store the snapshot for use, as is described in detail below. Alternatively, if the user does not feel that the captured snapshot is adequate, the user may once again select to capture a snapshot of the initial ultrasound scan (block 406). The option to load the captured snapshot is also provided by the ultrasound scanning unit 200 and displayed via the recording display 250.

It should be noted that if there is no initial rendering of an initial ultrasound scan, the user may still capture a single frame since typically an outline of the data portion 504 and video portion 502 of a rendered image may be seen regardless of whether data and video are rendered.

As is shown by block 410, the user may then physically select a region of the video portion 502 that will be used by the ultrasound recording unit 200, as is explained in detail below. Physical selection of the region of the video portion 502 may be performed in many different ways. As an example, the user may click a button on a mouse to select a portion of the video portion 502, and drag the mouse while holding down the button to select the region of the video portion 502 that will be used by the ultrasound recording unit 200.

The user is then provided with an option to save settings, specifically the physically selected region of the video portion 502, thereby completing configuration of the ultrasound recording unit 200 (block 412). In accordance with the first exemplary embodiment of the invention the selected region may be saved within the memory 220 (FIG. 4) as dimensions in pixels. Specifically, dimensions of the initial snapshot are known and the region that has not been physically selected, namely, the data portions 504, is subtracted from the initial snapshot, thereby resulting in the physically selected region, which is saved within the memory 220 (FIG. 4). Alternatively, the region may be saved as X pixels tall and Y pixels wide. The saved physically selected region of the video portion is the physical region of an ultrasound that will be provided in a finalized recording of an ultrasound, as performed by the ultrasound recording unit 200, as is explained in detail below.

In accordance with the second exemplary embodiment of the invention, the ultrasound recording unit 200 uses the machine display 310 to provide the above options to the user. In addition, the machine display 310 is used by the user to make selections offered by the ultrasound recording unit 200 and to display rendered and captured images, as has been described above.

While the abovementioned describes configuration of the ultrasound recording unit 200 prior to use, the ultrasound recording unit 200 may be pre-configured, thereby having the region of the video portion 502 already selected. In addition, the ultrasound recording unit 200 may have numerous options for regions of the video portion 502 listed for the user to select, where the regions may be based upon different manufactures of ultrasound machines 300 or machine displays 310. Alternatively, the ultrasound recording unit 200 may have a previous configuration stored within the memory 220 (FIG. 4) so that when the ultrasound recording unit 200 is initiated, the unit 200 automatically loads the last programmed region of the video portion 502, so as to remove the necessity for physically selecting the region for use by the ultrasound recording unit 200.

Ultrasound Recording

The following describes the process of recording an ultrasound in real-time, as is performed by the present recording system 100. It should be noted that, in practice, performance of the ultrasound is typically performed by a practicing physician that has knowledge of ultrasound procedures. Once configuration of the ultrasound recording unit 200 is complete, the recording display 250 is no longer needed. It should be noted that, alternatively, the recording display 250 may be kept on and images may be rendered thereto. Instead, the following displays and options may be displayed on the machine display 310, as instructed by the ultrasound recording unit 200. It should also be noted that the second indicator 204 (FIG. 3) continuously glows green to indicate that the ultrasound recording unit 200 is working.

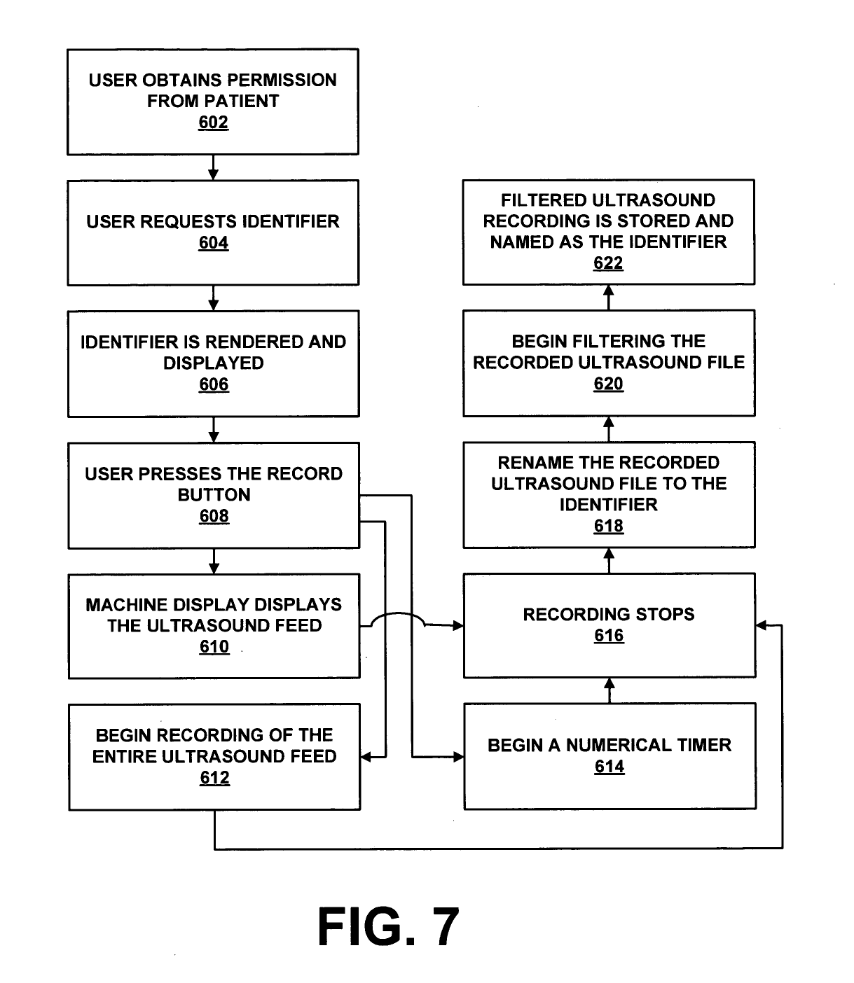

FIG. 7 is a flow chart illustrating the ultrasound recording procedure, in accordance with the first exemplary embodiment of the invention. As is shown by block 602, the user of the. ultrasound recording unit 200 first obtains permission from the pregnant woman (also referred to as a patient), or an associated party such as, but not limited to, her partner or parents, to record the ultrasound. By obtaining permission from the pregnant woman, the user of the ultrasound recording unit 200 prevents violation of any patient data confidentiality rights, as explained above.

As is shown by block 604, once the user obtains permission to record, the user requests an identifier. In accordance with the first exemplary embodiment of the invention, the user requests the identifier by pressing certain keys on a keyboard attached to the ultrasound machine 300. Of course, other methods may be used by the user to obtain the identifier. Preferably, the identifier is a series of characters that may be printed or written down by the user (the following assumes that the identifier needs to be written down). As an example, the identifier may be an eleven digit code where the first two digits represent the state in which the ultrasound is being performed, the next two digits represent the hospital in which the ultrasound is being performed, the next three digits represent the specific ultrasound recording unit 200 within the hospital, and the next four digits are a counter representing what number recording is presently being performed by the ultrasound recording unit 100.

It should be noted that in accordance with an alternative embodiment of the invention, the identifier may automatically be provided to the user of the ultrasound recording unit 200 once configuration is complete, or upon beginning an ultrasound.

The identifier is then rendered by the ultrasound recording unit 200 and displayed on the machine display 310 (block 606). The user of the ultrasound recording unit 200 may then write down the identifier and present the identifier to the pregnant women (block 606) so that she may later purchase or receive a finalized version of the recording performed by the ultrasound recording unit 200.

When the user is ready to begin the ultrasound clinical procedure, the user presses the record button 232 on the ultrasound recording unit 200 (block 608), which results in the first indicator 202 (i.e., the red LED) glowing red. While the first indicator 202 is glowing red, the second indicator 204 (i.e., the green LED) continues to glow green, thereby signifying that the ultrasound recording unit 200 is working properly. It should be noted that there need not be a record button 232 on the ultrasound recording unit 200. Alternatively, the ultrasound recording unit 200 may cause the machine display 310 to graphically present the option to begin recording to the user. The user may then use the keyboard or any other peripheral device connected to the ultrasound machine 300 to select to begin recording. In addition, if the machine display is a touch screen, the user may select to begin recording by simply touching the machine display 310 in a designated area.

After pressing the record button 232 (block 608), the ultrasound machine 300 causes the machine display 310 to display the ultrasound feed in real-time (block 610) so that the machine display 310 is displaying what is scanned by the ultrasound scanning device. After pressing the record button 232 (block 608) the ultrasound recording unit 200 begins recording the entire ultrasound feed from the ultrasound scanning device (block 612), as is displayed on the machine display 310. It should be noted that the recorded ultrasound feed includes both the data portion 504 and the video portion 502. Recording of the entire ultrasound feed is performed by storing the ultrasound feed within the storage device 218 for later use by the ultrasound recording unit 200, as explain below.

In addition, after pressing the record button 232 (block 608), the ultrasound recording unit 200 also begins a numerical timer (block 614) while recording the entire ultrasound feed. In accordance with the first exemplary embodiment of the invention the numerical timer is defined by the system software 222 stored within the ultrasound recording unit 200. The system software 222 defines a specific time span for continuous recording of the ultrasound feed. As an example, since a typical ultrasound procedure may last for approximately twelve minutes, the beginning five to eight minutes of scanning typically determines position of the baby and other factors that would results in images that are most ideal for recording. After the five to eight minutes the physician typically uses the ultrasound machine 300 to measure and locate different body parts of the baby so as to ensure proper development of the fetus. Preferably, since the later part of the ultrasound procedure (i.e., after the first five to eight minutes) is typically dominated by medical procedures, the first five to eight is selected for recording by the ultrasound recording unit 200.

It should be noted that use of the numerical timer is not a necessity. Alternatively, the user of the ultrasound recording unit may simply specify when to cease recording by using the ultrasound machine keyboard, a mouse, a touch screen option made available by the ultrasound recording unit 200, or any other means. However, use of the numerical timer allows the user to know beforehand what portions of the ultrasound feed will be recorded, thereby allowing the user to have a degree of control over what will later be available to the pregnant woman in the finalized version of the recording.

Once the numerical timer reaches the predefined time limit, the ultrasound recording unit 200 stops recording the ultrasound feed (block 616), thereby finalizing a recorded ultrasound file. The ultrasound recording unit 200 then renames the recorded ultrasound file to the identifier (block 618) that was previously provided to the pregnant woman.

After renaming of the recorded ultrasound file (block 618), the ultrasound recording unit 200 automatically begins filtering the recorded ultrasound file (block 620). Specifically, the recorded ultrasound file is filtered so that only the physically selected region of the video portion that was selected during configuration of the ultrasound recording unit 200 remains. The filtered ultrasound recording is then stored as a file within the storage device 218 and named as the identifier given to the pregnant woman (block 622). If the originally recorded ultrasound file is still located on the storage device 218, it is preferably deleted so as to save space on the storage device 218.

It should be noted that the original recorded ultrasound file and the filtered ultrasound file may be stored as different multimedia file types such as, but not limited to, moving pictures experts group files (MPEGs), audio/video interleave files (AVIs), Windows media files (WMVs), or any other multimedia file type.

Alternatively, filtering may be performed by the ultrasound recording unit 200 immediately prior to recording the ultrasound feed, so that the ultrasound is already filtered before being stored within the storage device 218. Filtering prior to storage decreases the amount the storage space required by the storage device 218 since a smaller area of the ultrasound feed is being stored. In addition, by filtering out data prior to storage, the present recording system 100 prevents violation of rules regulating the recording of medical data associated with the performed ultrasound.

Once the filtered ultrasound recording file has been stored the first indicator 202 (i.e., the red LED) stops glowing red and the second indicator 204 (i.e., the green LED) begins glowing green to indicate that the ultrasound recording unit 200 is ready to begin another recording session.

Optionally, if the user presses the record button 232 prior to completion of recording and/or filtering, the ultrasound recording unit 200 automatically deletes the ultrasound being recorded, or the filtered ultrasound being created. This gives the user of the ultrasound recording unit 200 a degree of control over whether the option to receive a copy of the filtered ultrasound will be provided to the pregnant woman.

In addition, the ultrasound recording unit 200 may provide a pause option that may be provided to the user. Use of the pause option would temporarily stop recording and/or filtering of the ultrasound, without resulting in deletion of the ultrasound being recorded or the filtered ultrasound being created.

It should be emphasized that the above-described embodiments of the present invention, particularly are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

What is claimed is:

1. A system for recording an ultrasound, comprising:
a memory; and
a processor configured by said memory to perform the steps of:
beginning recording of an entire ultrasound feed after an initiation to record;
ending said recording, thereby providing a recorded ultrasound file; and
filtering said recorded ultrasound file in accordance with a predefined region, thereby resulting in a filtered ultrasound file defined by said predefined region.

2. The system of claim 1, wherein said processor is further configured by said memory to perform the step of providing a user of said system with an identifier.

3. The system of claim 2, wherein said identifier is provided to a patient having said ultrasound.

4. The system of claim 2, wherein said processor is further configured by said memory to perform the steps of:
naming said recorded ultrasound file as said identifier;
naming said filtered recorded ultrasound file as said identifier; and
deleting said recorded ultrasound file.

5. The system of claim 1, wherein said processor is further configured by said memory to perform the steps of:
beginning a numerical timer after said initiation to record; and
performing said step of stopping recording at the end of a predefined time limit.

6. The system of claim 1, wherein said processor is further configured by said memory to perform the steps of:
stopping said step of recording if said user indicates to stop recording; and
deleting said recorded ultrasound file and said filtered ultrasound file if said system has not completed said step of filtering said recorded ultrasound file.

7. The system of claim 1, wherein said system is required to be configured prior to use, said configuration comprising the steps of:
rendering an initial ultrasound scan;
capturing a snapshot of said rendered ultrasound scan, wherein said snapshot comprises a video portion and a data portion; and
physically selecting a region of said video portion that will define a region of said video portion that is to be saved during said step of filtering said recorded ultrasound file.

8. The system of claim 7, wherein said configuration further comprises the step of loading said captured snapshot into said memory.

9. The system of claim 7, wherein said configuration is predefined so as to not require said rendering step, said capturing step, and said physically selecting step.

10. The system of claim 7, wherein said physically selected region of said video portion is saved in said memory as a prior configuration.

11. The system of claim 10, wherein said prior configuration is loaded during said configuration of said system, said prior configuration already having said region of said video portion that is to be saved during said step of filtering said recorded ultrasound file so that the step of physically selecting said region is not required.

12. A system for providing an ultrasound recording environment, comprising:
an ultrasound scanning machine having a machine display and an ultrasound scanning device; and
an ultrasound recording unit comprising:
a recording display;
a memory; and
a processor configured by said memory to perform the steps of:
beginning recording of an entire ultrasound feed after an initiation to record;
ending said recording, thereby providing a recorded ultrasound file; and
filtering said recorded ultrasound file in accordance with a predefined region, thereby resulting in a filtered ultrasound file defined by said predefined region.

13. The system of claim 12, wherein said entire ultrasound is a live ultrasound feed displayed on said machine display.

14. The system of claim 12, wherein said ultrasound recording unit further comprises a storage device for storing said recorded ultrasound file and said filtered ultrasound file.

15. The system of claim 12, wherein said ultrasound recording unit requires configuration prior to use, said configuration comprising the steps of:
rendering an initial ultrasound scan on said machine display;
capturing a snapshot of said rendered ultrasound scan, and displaying said captured snapshot on said recording display, wherein said snapshot comprises a video portion and a data portion; and
physically selecting a region of said video portion that will be saved during said step of filtering said recorded ultrasound file.

16. The system of claim 15, wherein said configuration is predefined so as to not require said rendering step, said capturing step, and said physically selecting step.

17. The system of claim 15, wherein a prior configuration is loaded during said configuration of said ultrasound recording unit, said prior configuration already having a region of said video portion that is to be saved during said step of filtering said recorded ultrasound file so that the step of physically selecting said region is not required.

18. A method of providing a recording of an ultrasound, comprising the steps of:
displaying a live ultrasound feed after being initiated to record by a user;
beginning recording of the entire ultrasound feed after said initiation to record;
ending said recording, thereby providing a recorded ultrasound file; and
filtering said recorded ultrasound file in accordance with a predefined region, thereby resulting in a filtered ultrasound file defined by said predefined region.

19. The method of claim 18, further comprising the step of providing an identifier.

20. The method of claim 19, further comprising the steps of:
naming said recorded ultrasound file as said identifier;
naming said filtered recorded ultrasound file as said identifier; and
deleting said recorded ultrasound file.

21. The method of claim 18, further comprising the steps of:
beginning a numerical timer after said initiation to record; and
performing said step of stopping recording at the end of a predefined time limit.

22. The method of claim 18, further comprising the steps of:
stopping said step of recording if an indication to stop recording is received; and
deleting said recorded ultrasound file and said filtered ultrasound file if said step of filtering said recorded ultrasound file has not been completed.

23. The method of claim 19, further comprising the step of providing said identifier to a patient.

24. The method of claim 18, further comprising the step of configuring for recording prior to said step of displaying said live ultrasound feed.

25. The method of claim 24, wherein said step of configuring further comprises the steps of:
rendering an initial ultrasound scan;
capturing a snapshot of said rendered ultrasound scan, wherein said snapshot comprises a video portion and a data portion; and
physically selecting a region of said video portion that will define a region of said video portion that is to be saved during said step of filtering said recorded ultrasound file.

26. The method of claim 25, wherein said configuration further comprises the step of loading said captured snapshot into a memory.

27. The method of claim 25, wherein said configuration is predefined so as to not require said rendering step, said capturing step, and said physically selecting step.

28. The method of claim 25, wherein a prior configuration is loaded during said step of configuring, said prior configuration already having a region of said video portion that is to be saved during said step of filtering said recorded ultrasound file so that the step of physically selecting said region is not required.
